(19) United States
(12) Patent Application Publication
(10) Pub. No.: US 2007/0017808 A1
(43) Pub. Date: Jan. 25, 2007

(54) Linear valve-coupled two-dimensional separation device and separation matrix and method

(75) Inventor: Xing Su, Cupertino, CA (US)

Correspondence Address: DLA PIPER US LLP, 4365 EXECUTIVE DRIVE, SUITE 1100, SAN DIEGO, CA, 92121-2133, US

(73) Assignee: INTEL CORPORATION, Santa Clara, CA (US)

(21) Appl. No.: 11/140,534

(22) Filed: May 27, 2005

(51) Int. Cl. C07K 1/26

(52) U.S. Cl. 204/450; 204/600

(57) ABSTRACT

A two-dimensional separation apparatus includes first and second modules operating respectively to separate a sample amount in a first dimension and a second dimension. A valve structure controllably isolates the first separation module from the second separation module.

### BACKGROUND

1. Field of the Invention

The invention relates to a biomolecule separation technique, and particularly to an apparatus and method for two-dimensional valve-controlled separation of biomolecules.

2. Description of the Related Art

Current techniques for separation of biomolecules, such as proteins and nucleic acids, and their analysis include polyacrylamide gel electrophoresis, size exclusion separation, affinity binding, salt precipitation, and HPLC separation, among others as understood by those skilled in the art (see, e.g., Thorsen et al, Microfluidic Large-Scale Integration, Science, Vol. 298, pp. 580-584, 18 Oct. 2002, which is hereby incorporated by reference). For analytical purposes, proteins in a biological sample are often separated based on either size or surface charges or hydrophilicity. Because many different proteins have similar sizes and/or hydrophilicities, such one dimensional separation is often not satisfactory.

To distinguish similar proteins, a two-dimensional separation technique may be used. For example, a protein species may be first separated in a device based on one property (e.g., iso-electro point, first dimensional separation) of the proteins and then separated again based on another property (e.g., size, second dimensional separation). The two-dimensional separation procedure of this example would however generally involve at least some tedious manual operation.

### BRIEF DESCRIPTION OF THE DRAWINGS

FIGS. 1A-1B schematically illustrate a linear valve-coupled two dimensional separation device in accordance with an embodiment of the invention.

FIGS. 2A-2B schematically illustrate a first separation module with post/pillar matrix in accordance with an embodiment of the invention.

FIGS. 3A-3B schematically illustrate a second dimensional separation module with electrode array matrix in accordance with an embodiment of the invention.

FIGS. 4A-4C schematically illustrate porous channels as separation matrix in accordance with an embodiment of the invention.

FIG. 5A schematically illustrates a side view of a two dimensional separation matrix in accordance with a preferred embodiment including a linear valve that is closed to initially isolate a first separation module from a second separation module.

FIG. 5B schematically illustrates a side view of the two dimensional separation matrix of FIG. 5A with the valve open.

FIG. 5C schematically illustrates a top view of the two dimensional separation matrix of FIGS. 5A-5B.

### DETAILED DESCRIPTION OF THE PREFERRED EMBODIMENTS

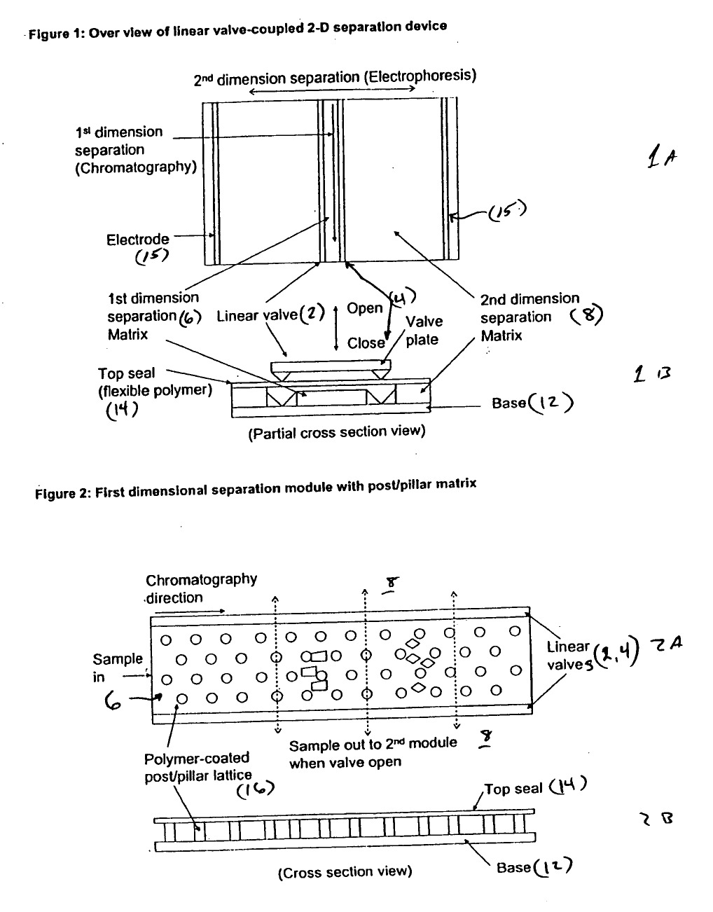

What follows is a description of one or more preferred and alternative embodiments of the invention. These embodiments will be described with reference to FIGS. 1A-5C. An apparatus is advantageously provided for two-dimensional separation of biomolecules. The apparatus includes first and second modules operating respectively to separate the biomolecules in a first dimension and a second dimension. A structure controllably isolates the first separation module from the second separation module. The structure preferably includes a valve. The valve may be a linear valve. The width is preferably substantially less than its length. The width of the linear valve may be less than 1/10 of its length. The structure may include two or more valves, and they may be linear valves.

Two or more separation devices are generally coupled together in accordance with a preferred embodiment through a strip-shaped linear valve. A commonly-used or unique disk-shaped valve may be changed or manufactured as a strip-shaped linear valve. This advantageous structure may be used to couple two or more separation modules.

An apparatus in accordance with a preferred embodiment for two-dimensional separation of biomolecules includes a base, a matrix layer, and a sealing layer. The matrix layer is formed on the base and includes first regions of a first separation configuration and second regions of a second separation configuration. The apparatus also includes a structure regulating migration of material between the first regions and the second regions.

A module for a two dimensional biomolecule separation apparatus in accordance with a preferred embodiment includes a structure for separating biomolecules. The structure may include a matrix of posts that interact with the biomolecules. The structure may include microchannels (e.g., having dimensions from 10 microns to 10 mm wide and from 1 cm to 50 cm long) having surfaces shaped to interact with selected characteristics of materials. The microchannels may include porous surfaces of selected pore size (e.g., from 10 nm to 100 microns).

### LINEAR VALVE-COUPLED SEPARATION MODULES

A valve is generally provided for a two dimensional biomolecule separation apparatus that includes at least first and second separation modules for separating biomolecules in at least two different dimensions. The valve includes a valve structure for controllably isolating the first separation module from the second separation module. The valve may include a linear valve wherein preferably the width of the linear valve is substantially less than its length, e.g., wherein the width is less than 1/10 of its length. The valve structure may include a unitary valve. The valve structure may include two valves that may be linear. The valve structure may include two linear valves. The valve structure may also include an elongated segment moveable between a first and a second position. The valve structure may also include an array of valves that may be positioned in a linear array.

Linear valves are used in accordance with a preferred embodiment to couple two-dimensional separation modules into a single device. FIGS. 1A and 1B are illustrative. Linear valves 2 and 4 are shown serving to permit material flow in a first direction when closed (e.g., from the top of the page to the bottom of the page for FIG. 1A and out of the page for FIG. 1B), and in a second direction when opened (e.g., to the left and to the right of valves 2 and 4 in FIGS. 1A and 1B). The valves 2, 4 are shown opening by moving upward in FIG. 1B and closing by moving downward in FIG. 1B. When closed, material may flow in the first dimension separation matrix 6 between the valves 2 and 4. When opened, the material may flow in the second dimension separation matrix 8 on either side of the matrix 6.

The linear valves 2 and 4 are long structures. Their length is preferably substantially longer than their width. For example, the length of the valves 2 may be more than ten times the width. A mechanism is provided that controls the opening and closing of the valves 2 and 4. Two parallel linear-valves 2 and 4 are located in the middle of the device illustrated at FIGS. 1A and 1B. When the valves 2 and 4 are closed, a channel-shaped separation module 6 (first dimensional separation module 6) is formed. The valves may be operated by pressure changes or other electrical or mechanical means. For example, for pressure-based operation, PDMS (poly-dimethyl silicon) channels may be used. As an example for electrical operation, a metal strip may be used and the operation of the valve may be based on thermal expansion (and contraction)

There are many alternative ways that could be used such as providing a system wherein opening the valves 2 and 4 allows the channel-shaped separation module to be formed, or wherein only a single valve is used which inhibits flow in a single direction, when closed, substantially orthogonal or at least different from the first dimensional flow. The width of the valve is preferably constant along its length, but it could vary. The material may be permitted to flow within the second dimension separation matrix first and then in the first afterward, although it is preferred to concentrate the material within the channel formed between the two proximate valves 2 and 4 first, and then permit the material to expand away from this channel 6 into the second dimension matrix 8.

Referring to FIG. 1B, the device preferably has three main layers: a base 12, a matrix layer including the first dimension matrix 6 and the second dimension matrix 8, and a top seal 14. The base 12 is preferably made of non-conducting materials, such as plastics or glass or PDMS. The regulating structure may be formed in the sealing layer and may include a MEMS (microelectromechanical system) valve. The regulating structure may also include an elongated structure formed between the first regions and the second regions that may include a portion shaped to receive a sealing structure and may further include an actuating portion for moving the sealing structure between a first position in which the sealing structure is received by the shaped portion and a second position in which a path is formed between the sealing structure and the shaped portion.

The matrix is the separation media 6 and 8. The matrix may include a uniform or gradient distribution of the posts. For example, structures having dimensions of 1 to 1000 microns in height and of 0.1 to 100 microns in diameter may be fabricated from silicon and can be coated with metal such as Au or Pt. The matrix may also include a distribution having a density at one end of the matrix that is larger than at the other end of the matrix. For example, the density of post may increase from 104 to 1010 posts per square cm. The posts may include a coated surface that facilitates the separating of the biomolecules. The posts may be embedded in a gel that facilitates the separating of the biomolecules. The posts may include an electrode array such that electrophoresis facilitates the separating of the biomolecules.

The top seal 14 confines the matrix space. It is preferably transparent and flexible, particularly in the region of the valves 2 and 4.

On both sides of the first dimensional separation module 6, there are preferably two flat second dimensional separation modules 8. In one embodiment, there are electrodes 15 located in parallel with the first module 6. After the first dimensional separation, molecules in this embodiment are moved laterally by electrophoresis into the second matrix 8 through the valves 2 and 4 when the valves 2 and 4 are opened.

### SEPARATION MATRIX

There are several optional matrix structures that can be used together with the linear structures 2, 4, or independent of the linear valve structures 2, 4. Different valve structures and/or separation matrices can be fabricated by standard photolithography techniques. A first example is illustrated at FIGS. 2A and 2B. The linear valves 2 and 4 are again shown for initially closing off the material within the first separation matrix 6 before opening and allowing the material to flow within the second separation matrix 8, in this case on either side of the channel 6. A matrix of posts 16 or pillars 16 are disposed within the first separation matrix 6.

The separation matrix 6 and/or 8 in accordance with this post/pillar 16 embodiment, preferably has the posts or pillars 16 fabricated from the base 12. The surfaces of the posts/pillars 16 of the matrix illustrated at FIG. 2A can be coated with an organic polymer, for example, hydrocarbon chain C18. Molecules in the sample may be separated based on their surface interaction with the matrix surfaces by liquid chromatography or other separation mechanism (e.g., electrophoresis).

Nano-structures may also be embedded in a gel matrix. The separation matrix 6 and/or 8 can also be made of nanostructures (posts or pillars 16) that are embedded in organic polymers. Optionally, a gradient of matrix density is used, with low density close to the first separation module 6. A gel matrix can comprise a linear acrylamide gel, a cross linked acrylamide gel, an agarose gel, or a gel formed by photo polymerization, among other gels that may be known to those skilled in the art.

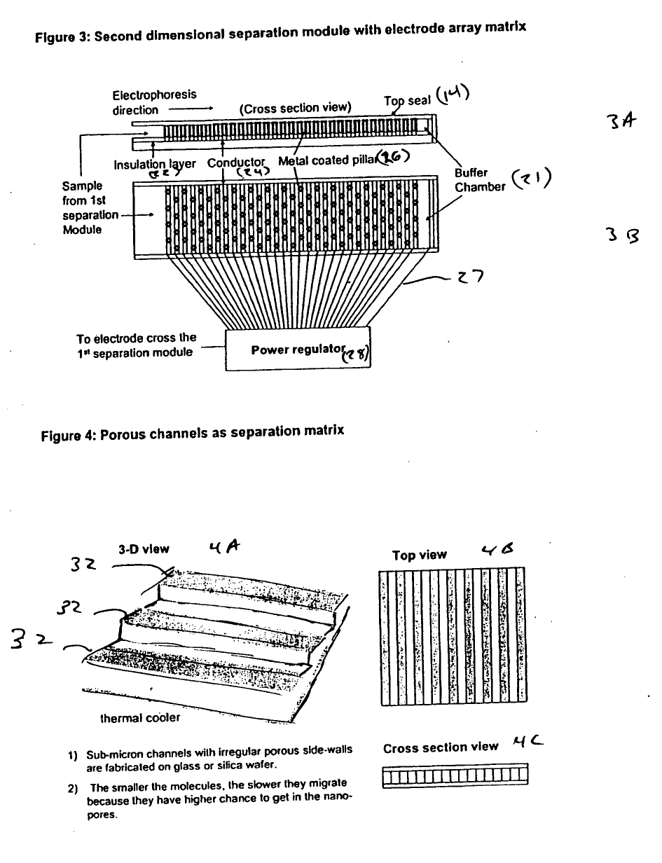

An electrode array matrix within a buffer chamber 21 is illustrated at FIGS. 3A and 3B. The apparatus includes a base insulation layer 22 and conductor layer 24 thereon. A matrix of metal- or otherwise conductor-coated pillars 26 are shown between the insulator and conductor layers and the top seal 14. ): The surfaces of the posts/pillars 26 can be coated with conductive metals (silver, gold) or semi conductive materials before molecule separation. As already illustrated at FIG. 1A, electrodes 15 may be preferably placed on peripheral sides of the modules 6, 8 (away from the first separation module 6). Optionally, many parallel conductors 27 can be connected to the metal coated posts 26 as illustrated at FIG. 3B. The multiple conductors are shown between a power regulator 28 in FIG. 3B and the posts/pillars 26. The electrode array can be used to sweep molecules away and/or attract molecules to their surfaces.

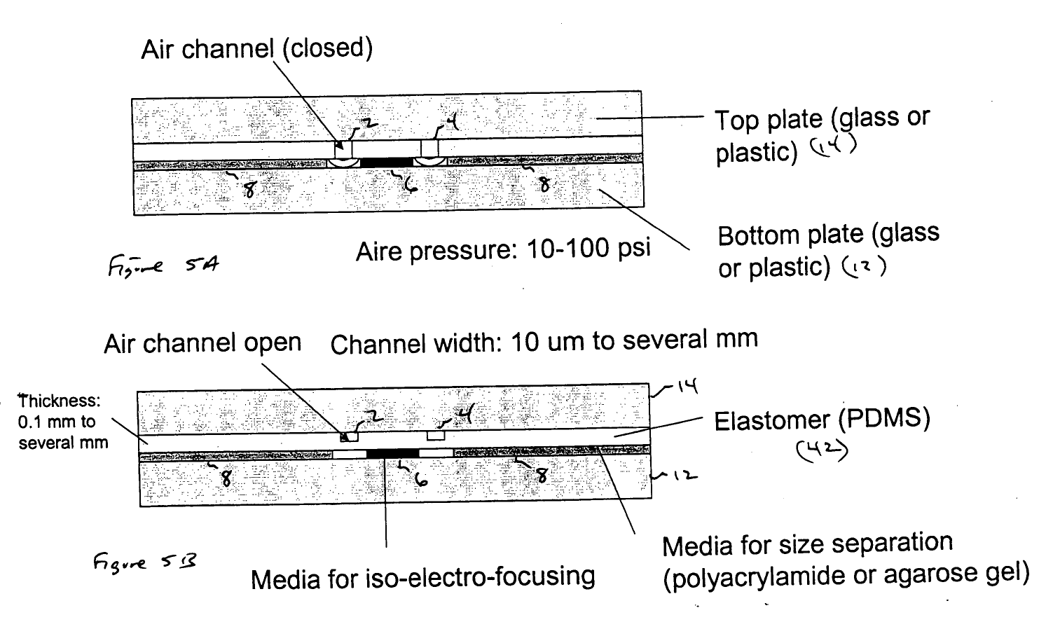

A porous channel matrix is illustrated at FIGS. 4A-4C. A matrix made of nano-channels with porous walls. When molecules are moved along the channels 32, small ones have a higher chance to get into the nanopores in the side walls and thus migrate slow in the separation direction. That is, sub-micron sized channels 32 with irregular porous side-walls are preferably fabricated on glass or a silica wafer. The smaller molecules with migrate more slowly than larger molecules, because they have a greater chance of becoming slowed by interaction with the nanopores.

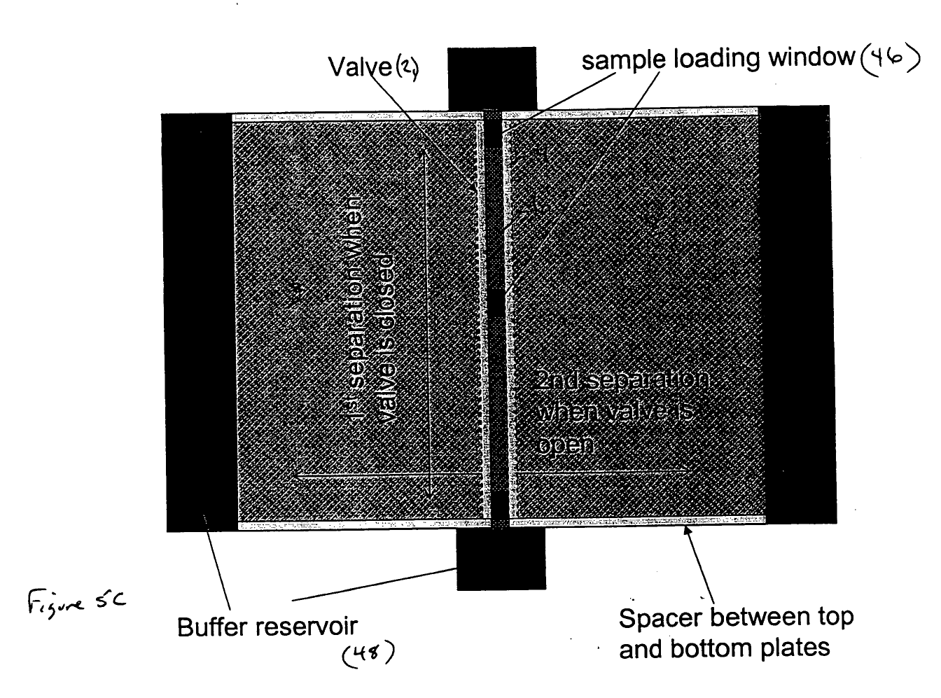

FIG. 5A schematically illustrates a side view of a two dimensional separation matrix in accordance with a preferred embodiment including a linear valve that is closed to initially isolate a first separation module from a second separation module. The module includes the linear valves 2 and 4 which selectively isolate a first dimensional separation matrix 6 from and second dimensional separation matrix 8. The sample matrix flows between a pair of plates 12, 14 that preferably comprise glass or plastic or another benign material known to those skilled in the art for the particular samples and separation matrices 6,8 that are being used. The material in this embodiment is thus physically compelled to move only in the planar region between the plates 12, 14, and the material is at least somewhat protected from the outer atmosphere. The vales 2, 4 of FIG. 5A are shown being subjected to increased pressure in order to close them, and the valves 2, 4 of FIG. 5B are shown being subjected to decreased pressure in order to open them. For example, a pressure over atmospheric pressure such as 100 psi may be applied to top of the valves 2, 4, according to the illustration of FIG. 5A, to close the valves 2,4, and either the valves 2,4 open automatically when the pressure is relieved (i.e., when the pressure is reduced back to atmospheric pressure, or about 14 psi), or a reduced pressure is applied such as less than atmospheric pressure such as 10 psi or less to open the valves. The process can work oppositely, wherein pressure is reduced at the top of valves 2, 4 to open them and increased to close them. The pressure changes can be applied to the top of the valves or to the bottom of the valves, as long as the relative pressure between the top and the bottom may be manipulated.

The valves 2, 4 may be alternatively operable mechanically, e.g., by lowering a lever, pushing a spring-loaded linear piece that is connected to the linear valves that may lock in place to close the valves 2, 4 and may be easily released so that the spring force lifts the piece and opens the valves 2, 4.

An electrical, battery powered or plug-in arrangement, e.g., such as a solenoidal-type of configuration may be used. A current may be selectively applied that creates a magnetic field which compels the valves 2, 4 to close. When the current is stopped or reversed, the valves 2, 4 open, or vice-versa. The solenoid magnet or magnet (not shown) may be perpendicularly arranged to the plane of the separation module and attached to the linear valves 2, 4. In this example, preferably at least two solenoids are used to stabilize the notion. Other mechanical, electrical, optical, or other arrangements or configurations may be understood by those skilled in the art for opening and closing the valves 2, 4.

FIG. 5B illustrates that an elastomer 42 such as PDMS (polydimethylsiloxane) may space the top and bottom plates 12, 14. The elastomer 42 would have a pair of linear channel to permit the valves 2, 4 to move slidably move therein. The space may preferably be a tenth of a millimeter or less or may be up to several millimeters depending on the viscosity of the sample and volume of the sample. The spacing may be adjustable, and the valves 2, 4 of sufficient size, to accommodate different samples and spacings. The width of the channels that form or accommodate the valves 2, 4 may be preferably between ten microns and several millimeters.

FIG. 5C schematically illustrates a top view of the two dimensional separation matrix of FIGS. 5A-5B. This view shows the valves 2, 4 and the separation matrices 6, 8, and also shows a trio of sample loading windows. There may a different number sample loading windows 46. When the sample is inserted via the windows 46 with the valves 2, 4 closed, the sample then spreads along the first separation matrix causing it to separation according to the mechanism of the first separation matrix. The flow of sample may be compelled by pressure, electrical or magnetic forces, gravity in some circumstances, or otherwise depending on the properties of the materials (e.g., a magnetic sample would respond to a magnetic field, a sample of large dimension may responds simply to gravity, etc.). There may be a buffer reservoir 48 that may be a single reservoir or dual reservoir, e.g., one or both dimensions or one for each dimension.

### SAMPLES

Molecules that can be separated by the device include proteins and protein derivatives (glycoproteins, phosphoproteins and lipidproteins) or nucleic acids. Protein complexes formed by non-covalent binding or nucleic acid-containing complexes can also be used.

According to one example, nano-barcode-probed target complexes or optical barcode-probed target complexes can be separated by the device. Nano-barcode generally refers to a signature related to the size or shape of the structure, while optical barcode generally refers to a signature related to photonic spectra. These terms can be used to describe a same structure. Optical and nano-barcode-probed target complexes include DNA, protein and/or molecular complexes. Properties of these complexes can be measured by fluorescence or Raman microscopy or using a scanning tunneling microscope (STM) or atomic force microscope (AFM). A certain selection, arrangement or configuration of the complex produces a unique spectrum or other signature when measured using any of these or other techniques known to those skilled in the art. The probes are typically parts of the complex and may include DNA or antibodies.

Other biomolecular samples that may be separated by the device include naturally occurring compounds, complexes of naturally-occurring compounds and synthetic compounds. Examples of naturally occurring compounds include amino acids, peptides, proteins, antibodies, nucleotides, oligo nucleotides, nucleic acid (DNA/RNA), sugar, polysaccharides, glycoprotein, lipid, lipid-proteins, metabolites, hormones, steroids, and vitamins. Examples of complexes of naturally occurring compounds include cells, bacterium, viruses, and any antigenic substances, that are made up of naturally-occurring compounds such as those listed above. Synthetic compound examples include genetically-engineered versions of any of the above, or chemically-modified versions such as synthetic peptides or synthetic oligo nucleotides.

### SAMPLES

Molecules that can be separated by the device include proteins and protein derivatives (glycoproteins, phosphoproteins and lipidproteins) or nucleic acids. Protein complexes formed by non-covalent binding or nucleic acid-containing complexes can also be used. For example, nano-barcode-probed target complexes or optical barcode-probed target complexes can be separated by the device. Other biomolecular samples that may be separated by the device include naturally occurring compounds, complexes of naturally-occurring compounds and synthetic compounds. Examples of naturally occurring compounds include amino acids, peptides, proteins, antibodies, nucleotides, oligo nucleotides, nucleic acid (DNA/RNA), sugar, polysaccharides, glycoprotein, lipid, lipid-proteins, metabolites, hormones, steroids, and vitamins. Examples of complexes of naturally occurring compounds include cells, bacterium, viruses, and any antigenic substances, that are made up of naturally-occurring compounds such as those listed above. Synthetic compound examples include genetically-engineered versions of any of the above, or chemically-modified versions such as synthetic peptides or synthetic oligo nucleotides.

### DETECTION

After separation, biomolecules can be detected by optical techniques, based on one or a combination of the following principles and/or groups of principles. First, absorption, reflection, polarization, and/or refraction may be used. Second, fluorescence or Raman and surface-enhanced Raman spectroscopy (SERS) may be used. Third, resonant light scattering (RLS) principles may be used. Fourth, grating-coupled surface plasmon resonance (GCSPR) techniques may be used.

While an exemplary drawings and specific embodiments of the present invention have been described and illustrated, it is to be understood that that the scope of the present invention is not to be limited to the particular embodiments discussed. Thus, the embodiments shall be regarded as illustrative rather than restrictive, and it should be understood that variations may be made in those embodiments by workers skilled in the arts without departing from the scope of the present invention as set forth in the appended claims and structural and functional equivalents thereof. For example, flexible polymer membranes may be used as valve material, such as may be in use or modified from those used in microfluidic systems. Further, many optical detection techniques are available and they can be applied to the integrated two-dimensional separation device and methods of the present invention. As to applications, the devices and methods of the invention can be used to make devices for clinical and bio-research applications.

A two-dimensional separation technique in accordance with a preferred embodiment makes it possible to integrate and miniaturize separation devices. It permits the integration and automation of two-dimensional separation procedures. It is particularly advantageous for biomolecular separation (for example, proteins). It saves reagents and shortens detection and/or diagnosis times. Clinical chemistry and biological research are greatly benefited, in the sense that separating a biological sample into individual molecular species for quality and quantity analysis may now be performed quickly and automatically.

In addition, in methods that may be performed according to preferred embodiments herein and that may have been described above, the operations have been described in selected typographical sequences. However, the sequences have been selected and so ordered for typographical convenience and are not intended to imply any particular order for performing the operations, except for those where a particular order may be expressly set forth or where those of ordinary skill in the art may deem a particular order to be necessary.

For example, a method of fabricating integrated devices for two-dimensional separation of biomolecules in accordance with a preferred embodiment includes coupling two or more separation modules together. The coupling includes a valve-coupling for valve-controlled separation of the biomolecules in a first dimension and a second dimension. In addition, a method of two-dimensional separation of biomolecules is also provided. The method utilizes a system of at least two separation modules that are valve-coupled for controllably isolating the at least two separation modules. The method includes separating the biomolecules in a first dimension corresponding to the valve-coupling of the at least two separation modules. The biomolecules are separated in a second dimension different from the first dimension, and the separating in the first and second dimensions is valve-controlled. In any of method or apparatus in accordance with a preferred embodiment, the separation in the first or second dimension, or both, may include applying pressure, a potential difference, and/or electrophoresis, and alternatively utilizing gravity if the sample dimension is suitably large, to facilitate biomolecule flow.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of fabricating integrated devices for two-dimensional separation of biomolecules, comprising coupling two or more separation modules together using a valve-coupling for valve-controlled separation of the biomolecules in a first dimension and a second dimension different than the first dimension.

2. The method of claim 1, wherein the separation in the first dimension comprises applying a potential difference to facilitate biomolecule flow.

3. The method of claim 1, wherein the separation in the first dimension comprises applying electrophoresis to facilitate biomolecule flow.

4. The method of claim 1, wherein the separation in the first dimension comprises applying pressure to facilitate biomolecule flow.

5. The method of claim 1, wherein the separation in the second dimension comprises applying a potential difference to facilitate biomolecule flow.

6. The method of claim 1, wherein the separation in the second dimension comprises applying electrophoresis to facilitate biomolecule flow.

7. The method of claim 1, wherein the separation in the second dimension comprises applying pressure to facilitate biomolecule flow.

8. An apparatus for two-dimensional separation of biomolecules, comprising:
a first module operating to separate the biomolecules in a first dimension;
a second module operating to separate the biomolecules in a second dimension different than the first dimension; and
a structure for controllably isolating the first separation module from the second separation module.

9. The apparatus of claim 8, wherein the first module is configured to facilitate material flow in the first dimension by application of pressure to facilitate biomolecule flow.

10. The apparatus of any of claims 9, wherein the second module is configured to facilitate material flow in the second dimension by application of pressure.

11. The apparatus of any of claims 9, wherein the second module is configured to facilitate material flow in the second dimension by application of a potential difference.

12. The apparatus of any of claims 9, wherein the second module is configured to facilitate material flow in the second dimension by electrophoresis.

13. The apparatus of claim 8, wherein the first module is configured to facilitate material flow in the first dimension by application of a potential difference.

14. The apparatus of any of claims 13, wherein the second module is configured to facilitate material flow in the second dimension by application of pressure.

15. The apparatus of any of claims 13, wherein the second module is configured to facilitate material flow in the second dimension by application of a potential difference.

16. The apparatus of any of claims 13, wherein the second module is configured to facilitate material flow in the second dimension by electrophoresis.

17. The apparatus of claim 8, wherein the first module is configured to facilitate material flow in the first dimension by electrophoresis.

18. The apparatus of any of claims 17, wherein the second module is configured to facilitate material flow in the second dimension by application of pressure.

19. The apparatus of any of claims 17, wherein the second module is configured to facilitate material flow in the second dimension by application of a potential difference.

20. The apparatus of any of claims 17, wherein the second module is configured to facilitate material flow in the second dimension by electrophoresis.

21. The apparatus of claim 8, wherein said structure comprises a valve.

22. The apparatus of claim 8, wherein the structure comprises a linear valve.

23. The apparatus of claim 22, wherein the width of the linear valve is substantially less than its length.

24. The apparatus of claim 22, wherein the width of the linear valve is less than 1/10 of its length.

25. The apparatus of claim 8, wherein the structure comprises two valves.

26. The apparatus of claim 8, wherein the structure comprises two linear valves.

27. A method of two-dimensional separation of biomolecules using a system of at least two separation modules that are valve-coupled for controllably isolating the at least two separation modules, comprising:
separating the biomolecules in a first dimension corresponding to the valve-coupling of the at least two separation modules; and
separating the biomolecules in a second dimension different from the first dimension, and wherein the separating in the first and second dimensions is valve-controlled.

28. The method of claim 27, wherein the separating in at least one of the first and second dimensions comprises applying a potential difference to facilitate biomolecule flow.

29. The method of claim 27, wherein the separating in at least one of the first and second dimensions comprises applying electrophoresis to facilitate biomolecule flow.

30. The method of claim 27, wherein the separating in at least one of the first and second dimensions comprises applying pressure to facilitate biomolecule flow.

31. An apparatus for two-dimensional separation of biomolecules, comprising:
a base;
a matrix layer formed on the base and comprising first regions of a first separation configuration and second regions of a second separation configuration;
a sealing layer; and
a structure regulating migration of material between the first regions and the second regions.

32. The apparatus of claim 31, wherein the base comprises a non-conducting material.

33. The apparatus of claim 31, wherein the regulating structure is formed in the sealing layer.

34. The apparatus of claim 31, wherein the regulating structure comprises a MEMS valve.

35. The apparatus of claim 31, wherein the regulating structure comprises an elongated structure formed between the first regions and the second regions.

36. The apparatus of claim 35, wherein the elongated structure comprises a portion shaped to receive a sealing structure.

37. The apparatus of claim 36, wherein the elongated structure further comprises an actuating portion for moving the sealing structure between a first position in which the sealing structure is received by the shaped portion and a second position in which a path is formed between the sealing structure and the shaped portion.

38. A module for a two dimensional biomolecule separation apparatus, comprising a structure for separating biomolecules in a first dimension, the structure comprising a matrix of posts that interact with the biomolecules.

39. The module of claim 38, wherein the matrix comprises a uniform distribution of the posts.

40. The module of claim 38, wherein the matrix comprises a gradient distribution of the posts.

41. The module of claim 38, wherein the matrix comprises a distribution having a density at one end of the matrix that is larger than at the other end of the matrix.

42. The module of claim 38, wherein the posts comprise a coated surface that facilitates the separating of the biomolecules.

43. The module of claim 38, wherein the posts are embedded in a gel that facilitates the separating of the biomolecules.

44. The module of claim 38, wherein the posts comprise an electrode array such that electrophoresis facilitates the separating of the biomolecules.

45. A module for a two dimensional biomolecule separation apparatus, the module comprising a structure for separating biomolecules in a first dimension, the structure comprising microchannels including surfaces shaped to interact with selected characteristics of materials.

46. The module of claim 45, wherein the microchannels comprise porous surfaces of selected pore size.

47. A valve for a two dimensional biomolecule separation apparatus that includes at least first and second separation modules for separating biomolecules in at least two different dimensions, the valve comprising a valve structure for controllably isolating the first separation module from the second separation module.

48. The valve of claim 47, wherein the valve structure comprises a linear valve.

49. The valve of claim 48, wherein the width of the linear valve is substantially less than its length.

50. The valve of claim 48, wherein the width of the linear valve is less than 1/10 of its length.

51. The valve of claim 47, wherein the valve structure comprises two valves.

52. The valve of claim 47, wherein the valve structure comprises two linear valves.

53. The valve of claim 47, wherein the valve structure comprises a unitary valve.

54. The valve of claim 47, wherein the valve structure comprises an elongated segment moveable between a first and a second position.

55. The valve of claim 47, wherein the valve structure comprises a plurality of valves positioned in a linear array.

56. The valve of claim 47, wherein the valve structure comprises an array of valves.
